(19) United States
(12) Patent Application Publication
(10) Pub. No.: US 2004/0109920 A1
(43) Pub. Date: Jun. 10, 2004

(54) Coated carotenoid cyclodextrin complexes

(75) Inventors: Helmut Reuscher, Onsted, MI (US); Daniel I. Kagan, Belmont, MA (US); Doddabele L. Madhavi, Worcester, MA (US)

Correspondence Address: BROOKS KUSHMAN P.C., 1000 TOWN CENTER, TWENTY-SECOND FLOOR, SOUTHFIELD, MI, 48075, US

(73) Assignees: BIOACTIVES LLC, Adrian, MI; WACKER BIOCHEM CORP., Adrian, MI

(21) Appl. No.: 10/309,999

(22) Filed: Dec. 4, 2002

(51) Int. Cl. A61K 31/724; A23L 1/30; A61K 9/16; A61K 9/50; A61K 31/015

(52) U.S. Cl. 426/73; 514/763; 514/58

(57) ABSTRACT

Coated cyclodextrin and carotenoid complexes are stable against oxidation and exhibit higher biouptake than oil-based, lipophile based, and micellular carotenoid compositions. The coating may be an oil, or a naturally occuring, optionally derivatized polymer or a pharmaceutically acceptable synthetic polymer.

### BACKGROUND OF THE INVENTION

1. Field of the Invention

The invention relates to a coated carotenoid cyclodextrin (CD) complex and a process for its production.

2. Background Art

The bioavailability of lutein and other carotenoids is known to be affected by dietary factors such as the composition of the carotenoid-rich food, the presence of adequate amounts of dietary fats and phospholipids, etc. For example, processing and cooking in the presence of vegetable oil has been shown to enhance the bioavailability of hydrocarbon carotenoids. D. A. Garrett, et al., “Estimation of carotenoid bioavailability from fresh stir-fried vegetables using an in vitro digestion/Caco-2 cell culture model”, J. NUTR. BIOCHEM., 11: 574-580, 2000; J. J. M. Castenmiller, et al., “The food matrix of spinach is a limiting factor in determining the bioavailability of β-carotene and to a lesser extent of lutein in humans”, J. NUTR., 129:349-355, 1999. Dietary fats and oils may facilitate the transfer of lutein, a lipophilic molecule, to the aqueous phase during digestion by the formation of micellarized lutein. The micelles in turn facilitate transfer of lutein to the brush border surface of enterocytes for uptake and subsequent absorption. Studies have also shown that the transfer of lutein to the micelles is affected by the digestive process and the presence of sufficient bile acids. D. A. Garrett, et al., op. cit. At present, lutein in supplements is delivered as an oil dispersion or as microencapsulated beadlets which require digestion and micellarization before absorption.

Cyclodextrins are cyclic oligosaccharides which consist of 6, 7 or 8 α(1-4)-linked anhydroglucose units. The α-, β- or γ-cyclodextrins, which are prepared by, for example, enzymatic starch conversion, differ in the diameter of their hydrophobic cavity and are generally suitable for the inclusion of a large number of lipophilic substances. Cyclodextrin is known to facilitate the transfer of molecules across the cellular membrane. Complexation of a lipophilic molecule with cyclodextrin also makes the compound dispersible in an aqueous phase in the absence of micellarization. In a recent study investigating the uptake of β-carotene and lutein by various cell membranes. I. Lancrajan, et al., “Carotenoid incorporation into natural membranes from artificial carriers: liposomes and betacyclodextrins”, CHEM. PHYS. LIPIDS, 112(1):1-10, 2001, the authors reported that uptake was increased by β-cyclodextrin inclusion complexes as compared with liposomal preparations.

Carotenoids are in general insoluble in water and are also sensitive to light, heat and oxidation. These properties, especially the water insolubility, give rise to poor bioavailability from pharmaceutical dosage forms such as capsules and tablets. Currently, the carotenoids are stabilized by encapsulation with a vegetable oil or with protective colloids. For example, United States Patent U.S. Pat. No. 5,976,575 discloses a dry carotenoid oil powder and a process for making the same. However, the encapsulation techniques does not ensure entry into the micellarization process or the uptake of the carotenoids in the intestine.

### SUMMARY OF THE INVENTION

It is an object of the present invention to both improve the stability of a carotenoid and to improve cellular uptake of the carotenoid into a cell. These and other objects are achieved by cyclodextrin carotenoid complexes which are subsequently coated with a bioacceptable organic coating material.

### BRIEF DESCRIPTION OF THE DRAWINGS

FIG. 1 illustrates the uptake of various forms of lutein by differentiated Caco-2 cells after 6 and 24 hours, in μg lutein per mg of cellular protein, as in Table 2.

### DETAILED DESCRIPTION OF THE PREFERRED EMBODIMENT(S)

The invention pertains to a cyclodextrin carotenoid complex furnished with a coating. The coated products exhibit excellent stability and biouptake.

The carotenoid may be any pharmaceutically or nutritionally acceptable carotenoid. Examples of carotenoids include astaxanthin, alpha-carotene, beta-apo-carotenol, beta-carotene, bixin, canthaxanthin, capsanthin, capsorubin, cryptoxanthin, fucoxanthin, lutein, lycopene, neoxanthin, violaxanthin, and zeaxanthin, as well as esters of carotenoids. Carotenoid extracts, crystalline carotenoids, or purified carotenoid powders may be used. The carotenoid is preferably selected among from lutein, astaxanthin, beta-carotene, and lycopene.

The cyclodextrin is preferably selected from among natural cyclodextrins and cyclodextrin derivatives, preferably alpha, beta and gamma cyclodextrin and hydroxypropyl cyclodextrins, especially gamma cylcodextrin and hydroxypropyl beta cyclodextrin.

The coating may be an oil, a natural polymer or a synthetic polymer. Derivatized oils and derivatized natural polymers may be used as well. The coating must be pharmaceutically and/or nutritionally acceptable, i.e. one which is unregulated or one which is approved by the FDA. The oil is preferably selected from among the vegetable oils, for example, castor, coconut, corn, cottonseed, evening primrose, flax, linseed, olive, palm, peanut, rapeseed (canola), safflower, sesame, soy, and sunflower oils, especially canola, corn and soy oils. Mixtures of oils may be used.

The natural polymer is preferably selected from among waxes (e.g. carnauba wax, candelilla wax, beeswax, rice bran wax, and paraffin wax), carbohydrates (e.g. cellulose, starch, pectin, alginate, carrageenan, furcellaran, chitosan), gums (e.g. gum arabic, gum xanthan, gum guar, gum ghatti, gum karaya, gum tragacanth, locust bean gum, gellan gum), resins (e.g. shellac, wood rosin, and tree resins such as copal, damar and elemi), proteins (e.g. fish and mammalian gelatin, soy protein, zein (from corn), casein, whey, wheat gluten, peanut protein), celluloses and cellulose derivatives (e.g. cellulose acetate, methyl cellulose, hydroxypropyl methyl cellulose), other polysaccharides (e.g. dextrin derivatives, alginates, pectins, starch derivatives) and especially Methocel™ celluloses and maltodextrin.

The natural polymers may be used in their native state (i.e. as isolated from natural sources), or may be chemically derivatized. Such chemical modification is well known, and includes, for example, sulfonation, phosphorylation, and carboxylation of natural polymers such as waxes, and especially chemical modification of polysaccharides, particularly celluloses. For example, the well known modified celluloses methyl cellulose, ethyl cellulose, propyl cellulose, hydroxymethyl cellulose, hydroxypropyl cellulose, etc., are obtained by such processes. Use of acetate cellulose derivatives of cellulose is also possible. Gelatin may be derivatized by reacting with aldehydes, for example formaldehyde, acetaldehyde, and propionaldehyde, which generally result in crosslinking of the gelatin.

The synthetic polymer is preferably selected from among the synthetic waxes such as polyethylene and polypropylene waxes, coumarene-indene resins, polylactic acid (PLA) and poly(lactic/glycolic) acid (PLGA), carylic polymers (methacrylic acid copolymers and ammonio methacrylate copolymers), polyorthoesters, polyphosphazenes, polyanhydrides, polyglycolide (PGA), poly(e-caprolactone), polydioxanone, trimethylene carbonate, poly(β-hydroxybutyrate), poly(g-ethyl glutamate), poly(DTH iminocarbonate), poly(bisphenol A iminocarbonate) and polycyanoacrylate, especially the acrylic polymers. Polyvinyl alcohol and polyvinylpyrollidone polymers may also be used.

A complex according to the invention preferably consists of gamma cyclodextrin, and preferably contains 5 to 25% by weight of a carotenoid. More preferred is a content of 5 to 10% by weight of a carotenoid.

The complexes according to the present invention can be used directly in the form of the reaction mixture. Alternatively, they can be isolated and processed by filtration, centrifugation, drying, grinding, sieving, screening, granulating or tableting to suit the procedure which is customary in each case.

The complexes can be used, for example, in the functional food or dietary supplements sector, and may be formulated alone or with other dietary ingredients.

The coated complexes according to the invention may be made by conventional microencapsulation techniques or by incorporation into hydrophobic oils. The coated products may be dried, if desired, by customary methods including, but not limited to, freeze drying and spray drying, preferably spray drying.

For example, the carotenoid and cyclodextrin complex (“complex”) may be prepared in a suitable medium, preferably water, to which an emulsion, dispersion, or solution of the coating component is then added. The coating component may coat or encapsulate the complex while in its preparation medium, or may do so all or in part during the drying process. Alternatively, the complex may be obtained in dry form, for example by tray drying, freeze drying, spray drying, etc., and then mixed with a solution, emulsion, or dispersion of the coating component followed by drying.

Other coating methods are suitable as well, including coacervation, prilling, compaction, agglomeration, fluidized spray drying, continuous fluidized bed granulation, rotor granulation, extrusion, submerged nozzle encapsulation, spray chilling, etc. For example, the complex, in dry form, may be introduced into a fluidized bed coater and coated therein. The coating component may be an oil, a solution, emulsion, or dispersion of a coating polymer, or in some cases, such as but not limited to waxy solids, may be a neat coating component. The coated complex may also be prepared by kneading the complex with a coating component, or by extrusion of the complex in admixture with a melt processable coating component or coextrusion of the complex with a melt processable coating component. Other coating methods will readily suggest themselves to one skilled in the art.

Spray drying is an especially useful method of preparing the coated complex formulations. Conventional spray dryers may be used, with single or multiple nozzles, disk atomizers, tangential spray, etc. Hot air flow may be concurrent or countercurrent, and spray may be top spray, wurster or bottom spray, tangential spray, etc.

The cyclodextrin carotenoid complex is made conventional cyclodextrin complexation techniques as known in the art, for example those disclosed in U.S. Pat. Nos. 4,777,162; 4,775,749; 4,831,022; and 5,189,149 and in published applications DE 196 12 658 A1 and CA 2,298,346.

The following preparation of carotenoid cyclodextrin complexes has proven advantageous: the carotenoid is added to an aqueous gamma cyclodextrin solution. The cyclodextrin concentration (“CD”) of the aqueous solution before the addition of the carotenoid is preferably between 30 and 50% by weight. A cyclodextrin concentration of 35-45% by weight is especially preferred. The weight ratio of carotenoid to CD is preferably between 1:3 and 1:20, more preferably between 1:9 and 1:20. The batches are mixed vigorously, i.e. kneaded or vigorously agitated, depending on their consistency.

The mixing of carotenoid and CD is preferably effected in a temperature range from 25 to 75° C., more preferably at 35 to 65° C., and most preferably at approximately 40 to 60° C. The mixing time depends on the temperature and is preferably between 0.5 and 1 hours. As a rule, a mixing time of 0.5 to 3 hours will suffice. However, the carotenoid and cyclodextrin mixture may be processed overnight with or without agitation following the mixing period. Complexing is preferably effected under atmospheric pressure, and preferably under a protective gas atmosphere of, for example nitrogen or argon. However, complexing may also be effected at higher or lower pressures as well.

The following examples are intended to illustrate the invention in greater detail, but should not be construed as limiting the scope of the invention in any way.

### EXAMPLES 1 - 3

### Complexation of Lutein with Cylcodextrins

Cyclodextrin inclusion complexes were prepared at several different concentrations, 20, 30 and 40% by weight. Dispersions in oil were prepared using the 30% complex (Example 2 below).

### Example 1

A 12% complex of Lutein-gamma cyclodextrin was prepared by dispersing 500g crystalline all-trans Lutein, from a commercially available source (BioActives LLC, Worcester, Mass., produced under U.S. Pat. No. 6,380,442) in 2,135g deionized, reverse osmosis (RO) filtered and degassed water. To this dispersion was added 2065.8g gamma cyclodextrin (8.82% moisture content). The mixture was stirred intensively for a minimum of 30 minutes. The observation of a viscosity increase during this time was a good indicator of favorable complexation conditions. The complex was allowed to stand overnight (˜20 hrs) and spray dried at 180° C. The yield was 1.5 Kg of lutein complex containing a calculated 12.0% lutein.

### Example 2

A 18% complex of Lutein-gamma cyclodextrin was prepared by dispersing 1000g crystalline Lutein in 4270g deionized, RO filtered and degassed water. To this dispersion was added 2376.3g gamma cyclodextrin (8.82% moisture content). The mixture was stirred intensively for a minimum of 30 minutes. The observation of a viscosity increase during this time was a good indicator of favorable complexation conditions. The complex was allowed to stand overnight (˜20 hrs) and spray dried at 180° C. The yield was 1.5 Kg of Lutein complex containing a calculated 18.0% Lutein.

### Example 3

A 23.6% complex of Lutein-gamma cyclodextrin was prepared by dispersing 500g Lutein in 2,135g deionized, RO filtered and degassed water. To this dispersion was added 780.9g gamma cyclodextrin (8.82% moisture content). The mixture was stirred intensively for a minimum of 30 minutes. The observation of a viscosity increase during this time was a good indicator of favorable complexation conditions. The complex was allowed to stand overnight ( 20 hrs) and spray dried at 180° C. The yield was 1.5 Kg of Lutein complex containing a calculated 23.6% Lutein.

### EXAMPLES 4-10

### Preparation of Coated Carotenoid/CD Complexes

### Example 4

A dispersion of 50% of the lutein complex of Example 2 was prepared in soy oil by mixing the complex into the oil for about 10 minutes. This yielded a stable dispersion with a calculated lutein content of 6.0%.

### Example 5

A dispersion of 60% of the lutein complex of Example 2 was prepared in soy oil by mixing the complex into the oil for about 10 minutes. This yielded a stable dispersion with a calculated lutein content of 10.8%.

### Example 6

A dispersion of 60% of the lutein complex of Example 2 was prepared in vegetable oil by mixing the complex into the oil for about 10 minutes. This yielded a stable dispersion with a calculated lutein content of 14.2%.

### Example 7

A cellulose coated 25% lutein complex was spray dried in a conventional spray drying apparatus having two spray nozzles, prepared using 560 grams of undried complex from Example 2 and Methocel® hydroxypropyl methylcellulose. The complex, at 30% solids, was sprayed at 5 ml/min from the top of the drying chamber and 5% Methocel E5 solution (50 grams in 1000 ml H₂O) was sprayed at 10 ml/min from the bottom of the drying chamber. The complex yield was about 70% overall. The product was a white powder coated at 30%, with 5.02% water content, giving an overall guest concentration of 25.8%.

### Example 8

A cellulose coated 25% lutein complex was prepared using 560 grams of undried complex from Example 2 and Methocel® hydroxypropyl methylcellulose. The, complex at 30% solids, was sprayed at 5 ml/min from the bottom of the drying chamber and 5% Methocel ES solution (50 grams in 1000ml H₂O) was sprayed at 10 ml/min from the top of the drying chamber. The complex yield was about 70% overall. The product was a white powder coated at 30%, with 5.12% water content, giving an overall guest concentration of 25.1%.

### Example 9

A cellulose coated 25% lutein complex was prepared using 500 grams of undried complex from Example 2 and Methocel® hydroxypropyl methylcellulose. The complex, at 30% solids, was sprayed at 5 ml/min from the top of the drying chamber and 10% Methocel ES solution (50 grams in 500 ml H₂O) was sprayed at 10 ml/min from the bottom of the drying chamber. The complex yield was about 70% overall. The product was a white powder coated at 30%, with 4.86% water content, giving an overall guest concentration of 26.0%.

### Example 10

A Cellulose coated 25% lutein complex was prepared using 200 grams of undried complex from Example 2, and Methocel® hydroxypropyl methylcellulose. 30% lutein Complex was dispersed in a 5% Methocel solution consisting of 50 grams Methocel E5 in 1000 ml H2O, a 25% coating ratio. The resulting product was spray dried. The resulting product was spray dried. Yield was about 65% overall. The product was a white powder with 4.84% water content, giving an overall guest concentration of 24.7%.

### EXAMPLES 11-12

### Stability and Bioavailability of Coated Carotenoid/CD Complexes

### Example 11

### Stability of the Lutein-cyclodextrin Complex

The samples used for testing were a lutein-cyclodextrin complex dispersion in soy oil containing 10.8% lutein (Example 5) and a commercially available 20% dispersion of free lutein in corn oil. The studies were done using approximately 5g of the dispersion in open vials. The vials were incubated at 55° C. in a forced air oven in the dark. The lutein content in the samples was analyzed by spectrophotometry after 14 days of incubation. Fourteen days under the conditions of this test roughly predicts the shelf-life of the product in two years at room temperature (U.S. Pat. No. 5,976,575). The results indicate that the lutein-cyclodextrin complex stability in corn oil over a period of fourteen days is comparable to the commercial sample (Table 1).

TABLE 1
Stability of lutein-cyclodextrin: Corn Oil Mixture

| | Day 0 | Day 14 |
|---|---|---|
| Sample | (wt. % lutein) | (wt. % lutein) |
| lutein-cyclodextrin | 10.68 | 10.00 |
| in oil | | |
| 20% lutein dispersion | 20.00 | 19.32 |

### Example 12

### Bioavailability of Lutein from Lutein-cyclodextrin Complex

The bioavailability of lutein was determined in vitro using Caco-2 human intestinal cells. This cell line has been used as a model to study the metabolism and transport of dietary carotenoids by a number of investigators: D.A. Garrett, et al., “Accumulation and retention of micellar b-carotene and lutein by Caco-2 human intestinal cells”, J. NUTR. BIOCHEM., 10:573-581, 1999; T. Sugawara, et al., “Lysophosphatidylcholine enhances carotenoid uptake from mixed micelles by Caco-2 human intestinal cells”, J. NUTR., 131:2921-2927, 2001. The cells spontaneously differentiate at confluency into cells that exhibit phenotypic properties that are similar to those of mature enterocytes, including a highly differentiated brush border. This cell line has also been used to examine the selective characteristics of intestinal absorption of the lipophilic nutrients vitamin E and retinol.

Materials: The culture medium and reagents were obtained from Sigma. A cyclodextrin-lutein complex dispersion in soy oil from example 5 was used for the studies. For comparison, micellarized lutein and a lutein-liposome were used. All manipulations were carried out under subdued light and the vials were covered with aluminum foil.

The stock solution of lutein-cyclodextrin complex was prepared by dispersing a sample of Example 5 (equivalent to 4mg lutein) in 10 ml phosphate buffered saline (PBS) using sonication. A stock solution of mixed micelles was prepared (Garrett, et al., 1999) using sodium taurocholate (bile salt) (0.914 mg), mono-acylglycerol (33 mg), oleic acid (94 mg), phosphatidyl choline (71 mg), lysophosphatidylcholine (71 mg) and lutein (4 mg) in 10 ml of PBS. The components were dissolved in dichloromethane, mixed at known concentrations and the solvent was evaporated using N₂. The residue was sonicated with 10 ml of PBS for three cycles of 5 min each with water changed after each cycle to minimize thermal degradation of lutein. Lutein-liposome stock solution was prepared using phosphatidylcholine (100 mg) and lutein (4 mg) in 10 ml of PBS. The components were dissolved in dichloromethane, mixed at specified concentrations, and the solvent was evaporated using N₂. The residue was sonicated in PBS as before to obtain the liposomes.

Method: Caco-2 (ATCC, Rockville, Md.) cells were maintained in high glucose DMEM with 15 mM HEPES and 10% heat inactivated fetal bovine serum, nonessential amino acids, glutamine and pyruvate in a humidified atmosphere at 5% CO₂ and 37° C. The cells were allowed to reach confluency (5-6 days after subculture) and differentiation (14 days) before the start of the experiment. The test samples were diluted in the culture medium to similar lutein concentrations for the experiment. The monolayers were washed with PBS before adding the test samples at known lutein concentration. The cultures were incubated as before for 6 and 24 hr. At indicated times, the medium was removed, the monolayers were washed with PBS, followed by three washes with 5 mM sodium taurocholate in PBS. The bile salt wash removes the lutein adhering to cell surfaces. The cells were scraped into cold PBS and pelleted using centrifugation. The lutein was extracted using cold methanol after a freeze-thaw cycle and estimated by spectrophotometry and HPLC.

The concentration of the test samples used was not cytotoxic to the cells as determined by the gross morphological appearance of the monolayers, and the total protein content per flask was similar in cultures incubated with and without the test samples.

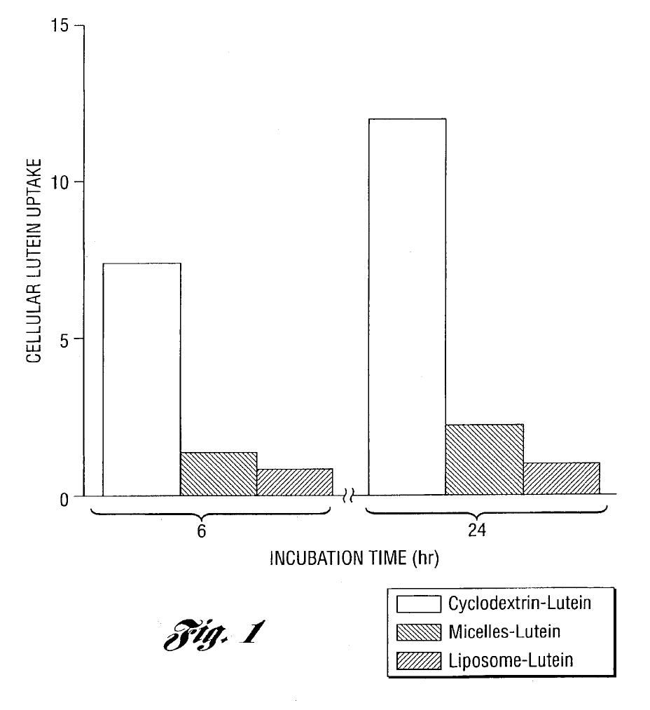

Observations: The results indicate that the lutein uptake from the cyclodextrin complex is nearly 5 to 10 fold higher as compared to the mixed micelles and the liposomes (FIG. 1 and Table 2). The cyclodextrin complexation enhanced lutein uptake by the cells even in the absence of micellarization or phospholipids.

TABLE 2
Uptake of Lutein by Differentiated Caco-2 Cells

| | Cellular Lutein | Cellular Lutein |
|---|---|---|
| | [microgram/milligram cellular | [microgram/milligram |
| | protein] | cellular protein] |
| Lutein sample | (Incubation Time 6 h) | (Incubation Time 24 h) |
| Coated | 7.45 | 12 |
| Cyclodextrin- | | |
| Lutein | | |
| Micelles- | 1.4 | 2.16 |
| Lutein | | |
| Liposome- | 0.86 | 0.86 |
| Lutein | | |

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A coated carotenoid formulation comprising one or more carotenoids in a cyclodextrin or cyclodextrin derivative, said complex coated with a pharmaceutically acceptable coating agent.

2. The coated carotenoid formulation of claim 1, wherein said coating agent is selected from the group consisting of optionally derviatized natural oils, optionally derivatized natural polymers, synthetic polymers, and mixtures thereof.

3. The formulation of claim 1 wherein the coating comprises an oil derived from vegetable sources.

4. The formulation of claim 1 wherein the coating comprises an optionally derivatized natural polymer.

5. The formulation of claim 1 wherein the coating is a synthetic polymer.

6. The formulation of claim 1, wherein the cyclodextrin is selected from the group consisting of α-cyclodextrin, βP-cyclodextrin, γ-cyclodextrin, and mixtures thereof.

7. The formulation of claim 1, wherein the carotenoid is selected from the group consisting of lutein, astaxanthin, β-carotene, and lycopene.

8. The formulation of claim 1, wherein the complex comprises 5-25% by weight of a carotenoid based on the weight of the carotenoid and the cyclodextrin.

9. The complex of claim 2, wherein at least one oil is selected from the group consisting of soy oil, canola oil, and corn oil.

10. The complex of claim 4, wherein said natural polymer comprises a cellulose or cellulose derivative.

11. A process of preparing the coated carotenoid formulation of claim 1, comprising:
a) preparing a complex of carotenoid in a cyclodextrin; and
b) coating said complex with a coating component comprising an optionally derivatized natural oil, an optionally derivatized natural polymer, or a synthetic polymer.

12. The process of claim 11 wherein said complex is present as a discontinuous phase in a dispersion of said complex, a polymer coating component comprising a solution, emulsion, or dispersion of an optionally derivatized natural polymer, a synthetic polymer, or mixture thereof is added to said dispersion of said complex to form a complex and coating component mixture, and drying said mixture to form a polymer-coated carotenoid cyclodextrin complex.

13. The process of claim 12, wherein said dispersion of said complex is an aqueous dispersion.

14. The process of claim 13, wherein said polymer coating component comprises a water soluble polymer.

15. The process of claim 11 wherein following step b) a dispersion of a carotenoid complex coated with a polymer or a dispersion of a carotenoid complex containing a coating component is obtained, and said dispersion is dried.

16. The process of claim 15, wherein said dispersion is freeze dried or spray dried.

17. A process for the preparation of the coated carotenoid formulation of claim 1, comprising:
a) preparing a complex of carotenoid in a cyclodextrin;
b) drying said complex;
c) extruding said complex together with a polymer coating component to form a polymer coated complex extrudate.

18. A process for the preparation of the coated carotenoid formulation of claim 1, comprising:
a) preparing a complex of carotenoid in a cyclodextrin as a dispersion in a liquid continuous phase;
b) providing a second liquid phase containing a polymer coating component; and
c) contacting said dispersion a) with said liquid phase b) in a spray drier and drying to obtain a dry polymer-coated carotenoid cyclodextrin formulation.

19. A process for the preparation of the coated carotenoid formulation of claim 1, comprising:
a) preparing a complex of carotenoid in a cyclodextrin;
b) introducing said complex into a fluidized bed coating apparatus together with a coating component; and
c) removing from said fluidized bed coating apparatus a dry, coated carotenoid cyclodextrin formulation.

20. The process of claim 12, comprising:
a) preparing a carotenoid and cyclodextrin complex in aqueous medium;
b) adding to said aqueous medium a soluble polysaccharide coating polymer to form a coating-component-containing carotenoid cyclodextrin complex mixture; and
c) drying said mixture.

21. The process of claim 20 wherein said step of drying comprises spray drying.

22. The process of claim 21 wherein said polysaccharide comprises at least one optionally derivatized cellulose.

23. The process of claim 11, comprising:
a) preparing a carotenoid and cyclodextrin complex in aqueous medium;
b) preparing an aqueous composition of a polysaccharide; and
c) introducing said carotenoid and cyclodextrin complex in aqueous medium and said aqueous composition of a polysaccharide into a spray dryer, and drying to obtain a coated carotenoid cyclodextrin formulation.

24. The process of claim 23 wherein said polysaccharide comprises at least one optionally derivatized cellulose.
